(19) United States
(12) Patent Application Publication
(10) Pub. No.: US 2026/0183133 A1
(43) Pub. Date: Jul. 2, 2026

(54) CAST PRESSURE RELIEVING SYSTEM

(71) Applicants: Hani Haider, Carter Lake, IA (US); Matthew A Halanski, Scottsdale, AZ (US)

(72) Inventors: Hani Haider, Carter Lake, IA (US); Matthew A Halanski, Scottsdale, AZ (US)

(21) Appl. No.: 19/438,704

(22) Filed: Jan. 2, 2026

Related U.S. Application Data
(60) Provisional application No. 63/741,214, filed on Jan. 2, 2025.

(51) Int. Cl. A61F 5/058

(52) U.S. Cl. CPC A61F 5/0585

(57) ABSTRACT

A system and method for mitigating pressure sores in patients subjected to cast immobilization. The system comprises an inflatable bladder with a filling port extending to the outside of the cast or the cast is cast to set around it, at least one wing for affixing the bladder to a patient's region, and a pocket formed by the bladder when inflated. The bladder is designed to prevent, contact between the cast material and the patient's region during the casting process, leaving a void. The bladder can be inflated with a fluid medium such as air, water or alcohol. The system may also include an adhesive indicator strip with graduated markings for monitoring the foot's position within the cast, and a pressure detection system with sensors configured to detect high-pressure areas and communicate with a central control unit or mobile device application.

### CROSS REFERENCE TO RELATED APPLICATIONS

This application claims benefit from currently pending U.S. Provisional Application No. 63/741,214 titled “Cast Pressure Relieving System” and having a filing date of Jan. 2, 2025, all of which is incorporated by reference herein.

### FIELD OF THE INVENTION

The present invention pertains to medical devices and orthopedic care, specifically focusing on mitigating pressure sores in patients undergoing cast immobilization due to fractures or congenital deformities and more specifically a cast pressure relieving system for pressure detection, monitoring, and alleviation tissue injury from orthopedic casts.

### BACKGROUND OF THE INVENTION

Pressure sores, also known as decubitus ulcers or pressure ulcers, represent a significant clinical challenge in orthopedic care, particularly for pediatric patients subjected to cast immobilization. These lesions develop as a result of prolonged tissue compression and compromised blood circulation, leading to tissue ischemia and eventual necrosis. Pressure sores most commonly develop on skin overlying bony prominences, including the heels, ankles, elbows, knees, head of the fibula, sacrum, and other anatomical locations where bones lie close to the skin surface with minimal soft tissue cushioning.

The etiology of pressure sores in cast-immobilized patients is multifactorial and largely associated with unrelieved mechanical pressure exerted by rigid cast structures against the patient's skin. This pressure is particularly concentrated in the heel region and other bony prominences, where the anatomical geometry creates localized stress concentrations. The problem is compounded by the patient's limited mobility and inability to naturally adjust their positioning within the cast, resulting in sustained pressure that exceeds the capillary perfusion pressure threshold, thereby causing tissue damage.

Conventional methods for reducing the risk of pressure sores in pediatric and adult patients often rely on the application of additional soft padding materials beneath the cast, such as cotton batting, synthetic fiber wadding, felt pads, or foam inserts. These conventional padding approaches, however, have significant inherent limitations that compromise their effectiveness. While such padding materials can provide an initial degree of cushioning effect, they fail to address the fundamental problem of pressure redistribution over time and often lead to an imperfect anatomical fit that does not adequately accommodate individualized patient anatomy or the dynamic movements that occur within a cast.

Specifically, the application of cast material around conventional padding tends to compress the padding material, thereby reducing or eliminating its cushioning effect. Furthermore, patient movement within the cast, particularly plantar flexion and dorsiflexion of the foot, can cause the heel to compress the padding through mechanical action, resulting in padding consolidation. Even when padding compression is relatively lower compared to unpadded casting, the residual pressure from the compressed padding material itself can still exceed safe thresholds and contribute to pressure sore formation. Consequently, static padding alone does not adequately mitigate the risk of pressure sores, as it provides no mechanism to prevent the foot or other body part from slipping or translating within the cast, which can exacerbate localized pressure points and undermine the intended protective effect.

Existing technologies in the orthopedic field, which may involve static spacer devices or cushioning materials positioned within a cast to create nominal space, lack dynamic pressure monitoring capabilities and fail to provide effective pressure management over the duration of cast immobilization. These prior art approaches do not offer practical solutions for detecting and responding to changes in patient positioning that could precipitate pressure sore development. Additionally, current systems lack the ability to detect high-pressure areas within the cast in real-time, meaning that caregivers often do not realize that a pressure sore is developing until visible tissue damage has already occurred, at which point treatment becomes more complex, costly, and the patient's discomfort and morbidity are significantly increased.

Therefore, there is a need for a device and method that can effectively mitigate pressure sores in pediatric patients subjected to cast immobilization wherein such a system should encompass mechanisms for both proactive and reactive interventions to alleviate pressure in critical areas while accommodating individual patient anatomy and mobility within a cast.

### SUMMARY OF THE INVENTION

The present invention provides a heel pressure relieving system for a patient receiving a rigid cast formed of casting materials in a casting procedure. The system can comprise a bladder having an extended filling port, an interior cavity and an exterior surface. The system can comprise at least one wing coupled to the exterior surface of the bladder, wherein the at least one wing can be configured to removably affix the bladder to a skin surface of a body region of the patient. The extended filling port can allow the bladder to receive a fluid to selectively inflate the bladder from a collapsed configuration to an expanded configuration and can allow a user to release the fluid through the filling port after the cast material has cured to form the rigid cast.

The bladder can be made from material selected from rubber, latex, thermoplastic polyurethane, neoprene rubber, butyl rubber, or nitrile. The bladder can be an elongated shape configured to conform to a body region of the patient. The at least one wing can comprise at least one vertical wing and at least one horizontal wing, where each wing can be configured to removably couple to the patient's skin through an adhesive backing. The body region can comprise a heel, an ankle, an elbow, a knee, a head of a fibula, a forearm, or a sacrum.

The fluid medium can comprise air, water, alcohol, gel, nitrogen, or carbon dioxide. The system can further comprise an indicator strip configured to be applied to a surface of the patient's body, where the indicator strip can have graduated visual markings that can provide a visual indication of the body region's position relative to the rigid cast. The filling port can comprise a valve configured to retain the fluid medium within the interior cavity during the casting procedure and to permit controlled release of the fluid medium after the cast material has cured.

The extended filling port can comprise a tubular neck section extending from the bladder, where the tubular neck section can be configured to extend through an aperture in the rigid cast to provide external access for inflation and deflation. The system can further comprise a rigid ring structure about the tubular neck section configured to be embedded in the cast material during curing, where the rigid ring structure can form a defined aperture through which the bladder can be removed after deflation. The system can further comprise a protective cover encapsulating at least a portion of the exterior surface of the bladder, where the protective cover can comprise nylon fabric, polyurethane-coated fabric, vinyl coating, polyester fabric, mesh material, spandex, or combinations thereof. The fluid can be colored to provide a visual indicator of the configuration of the bladder.

In another aspect, the invention provides a system for mitigating pressure sores in a patient subjected to cast immobilization. The system can comprise a bladder configured to form a protective air pocket around a body region of the patient within a cast structure. The system can comprise a plurality of pressure sensors embedded within or adjacent to the bladder, where the plurality of pressure sensors can be configured to detect pressure levels between the patient's skin and the cast structure. The system can comprise a control unit in communication with the plurality of pressure sensors, where the control unit can be configured to receive pressure data from the plurality of pressure sensors, compare the pressure data to a predetermined pressure threshold and generate an alert when the pressure data exceeds the predetermined pressure threshold. The control unit can communicate with a mobile device application through a wireless communication protocol such as Bluetooth, Wi-Fi, cellular communication, or proprietary radio frequency protocols. The plurality of pressure sensors can comprise capacitive pressure sensors, resistive pressure sensors, piezoelectric sensors, piezoresistive sensors, optical pressure sensors, or combinations thereof.

In yet another aspect, the invention provides a method for mitigating pressure sores during cast immobilization of a patient. The method can comprise securing a bladder to a skin surface of a body region of the patient using at least one adhesive wing. The method can comprise inflating the bladder with a fluid medium to create an expanded configuration during a casting procedure. The method can comprise applying cast material that cures to achieve structural rigidity around the bladder in the expanded configuration. The method can comprise allowing the cast material to cure and achieve structural rigidity. The method can comprise deflating the bladder after the cast material has cured, thereby creating a persistent air pocket between a rigid cast structure and the skin surface.

Deflating the bladder can comprise cutting a filling port of the bladder, opening a valve mechanism, puncturing the bladder through a cast tube, or untying a knot sealing the filling port. The method can further comprise applying an adhesive indicator strip to the skin surface of the patient before applying the cast material, where the indicator strip can have graduated markings to provide visual monitoring of a position of the body region during the casting procedure. The method can further comprise adjusting an inflation state of the bladder after cast material has been applied but before the cast material achieves structural rigidity by adding additional fluid medium to increase inflation and/or releasing fluid medium to decrease inflation. The method can further comprise removing the bladder through an aperture in the rigid cast structure after deflation.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors'intent and desire that the simple, plain and ordinary meaning to the terms be applied to the interpretation of the specification and claims. Aspects and applications of the invention presented here are described below in the drawings and detailed description of the invention.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of . . . ”), without also reciting in such phrases any structure, material or act in support of the function. Thus, even when the claims recite a “means for performing the function of . . .” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF DRAWINGS

A more complete understanding of the present invention may be derived by referring to the detailed description when considered in connection with the following illustrative figures. In the figures, like reference numbers refer to like elements or acts throughout the figures.

FIG. 1 shows a side view of cast pressure relieving system in accordance to one or more embodiments;

FIG. 2 shows a back view of cast pressure relieving system omitting the foot and cast in accordance to one or more embodiments;

FIG. 3 shows a heel load on a well-fitted cast with different interventions in accordance to one or more embodiments;

FIG. 4 shows a load measure at heel with and without the heel pressure relieving system in accordance to one or more embodiments;

FIG. 5 shows an example embodiment on a patient in accordance to one or more embodiments;

FIG. 6 shows a front view of cast pressure relieving system omitting the foot and cast in accordance to one or more embodiments; and

FIG. 7 shows a side view of another embodiment of the cast pressure relieving system in accordance to one or more embodiments.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth to provide a thorough understanding of the various aspects of the invention. It will be understood, however, by those skilled in the relevant arts, that the present invention may be practiced without these specific details. In other instances, known structures and devices are shown or discussed more generally to avoid obscuring the invention. In many cases, a description of the operation is sufficient to enable one to implement the various forms of the invention, particularly when the operation is to be implemented in software. It should be noted that there are many different and alternative configurations, devices, and technologies to which the disclosed inventions may be applied. The full scope of the inventions is not limited to the examples that are described below.

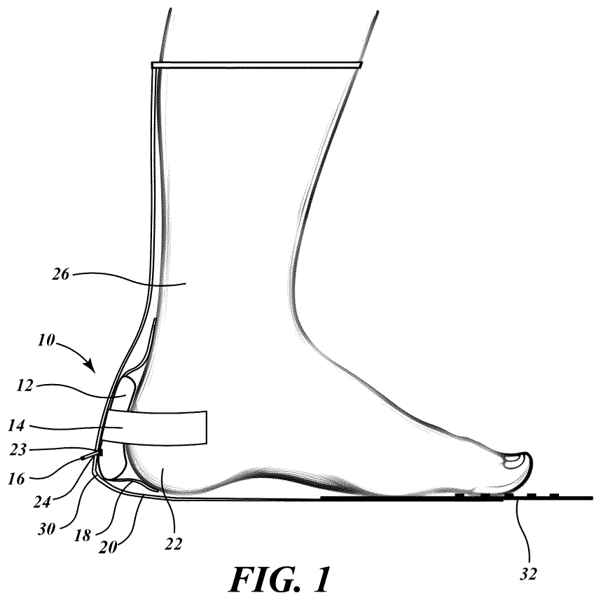

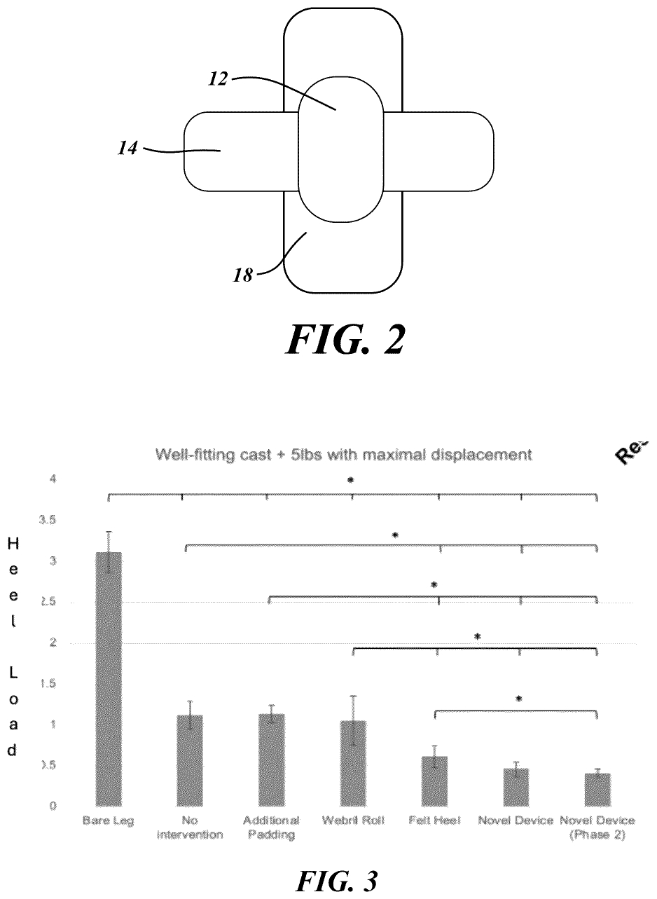

Referring initially to FIG. 1-2 a cast pressure relieving system is shown generally at 10 where in the system is designed to mitigate pressure sores on a patient subjected to cast immobilization. The cast pressure relieving system can be applied anywhere on the patient that has high pressure sensitive areas such as, for example, the heel, ankle, elbow, knee, head of the fibula, forearm, sacrum, or the like.

In embodiments, the cast pressure relieving system 10 can comprise a bladder 12 and can have a narrow neck leading to a filling port 16. The bladder 12 can be any suitable shape or size to fit the high-pressure sensitive area on the patient and in the preferred embodiment for the heel, the bladder can be an elongated shape and vary in size to fit different patient's heel 22 size. The bladder 12 can be made from such as, for example, silicone, latex, thermoplastic polyurethane, neoprene, butyl rubber, nitrile rubber, elastic material, or the like. The bladder 12 can be, such as, for example, air bladder, alcohol bladder, water bladder, pneumatic bladder, or the like.

The bladder 12 can be inflated with a fluid medium wherein the fluid medium can be such as, for example, a liquid, +/− color agent, water, alcohol, gel, air, gas, powder, mild sterilizing liquid, antimicrobial solution, or the like providing a safe and effective means of inflation. The fluid medium can be a color allowing the user to confirm that the bladder is decompressed when the cast is hardened. The bladder 12 can be inflated either before placement in the cast or can inflated before the cast sets or can be fined tuned by pumping air into the bladder or releasing air from the bladder before the cast placement or before the cast sets. The bladder 12 can have a cover encapsulating it or the bladder can be coated with such as, for example, nylon fabric, polyurethane-coated fabric, vinyl coating, polyester fabric, mesh cover, spandex cover or the like. The covering can be permanently bonded to the bladder surface through such as, for example, adhesive lamination, heat bonding, encapsulation molding or the like. In other embodiments, the covering can be omitted entirely, and the inflatable bladder 12 can be placed in direct contact with the patient's skin 26 without any intervening material layer.

In embodiments, the bladder 12 can have a wall thickness that can range from approximately 0.2 mm to 10.0 mm, with preferred embodiments using wall thickness in the range of 0.5 mm to 3.5 mm, or the like. The bladder 12 material can be manufactured by such as, for example, dip molding, injection molding, blow molding, vacuum forming processes, or the like. In certain embodiments, the bladder 12 can be designed as an air bladder inflated with atmospheric air, compressed air, or other gases such as nitrogen or carbon dioxide. In other embodiments, the bladder 12 can be configured as a liquid-filled bladder that can be inflated with water, sterile saline solution, alcohol such as isopropyl alcohol or ethanol, or other biocompatible liquids.

In embodiments, the bladder 12 can be coupled to at least one wing 15, which can be used to affix the bladder to the heel region of the patient. The at least one wing 15 can be a vertical wing 18 and a horizontal wing 14. The vertical wing 18 can be removably attached to the upper and lower portion of the patient's heel 22 and the horizontal wing 14 can be removably attached to both the inner and outer portion of the user's heel. The vertical wing 18 can extend from the bladder body in a direction generally parallel to the long axis of the leg, and can have dimensions typically ranging from such as, for example, approximately 20 mm to 120 mm in length and approximately 15 mm to 100 mm in width. The horizontal wing 14 can be configured to removably attach to both the medial and lateral aspects of the user's heel, providing horizontal positional stability and resistance to medial-lateral displacement. The horizontal wing 14 can extend from the bladder body in a direction generally perpendicular to the long axis of the leg and can have similar dimensional ranges to the vertical wing 18.

In other embodiments, the at least one wing 15 can be different configurations beyond the horizontal and vertical arrangement such as, for example, the adhesive wings can be positioned horizontally only omitting the vertical component, vertically only omitting the horizontal component, or can be arranged in diagonal orientations relative to the bladder body. In other embodiments, the at least one wing 15 can have three or more wing members arranged radially around the bladder perimeter, providing 360-degree circumferential attachment stability. In embodiments, the at least one wing 15 can be manufactured from such as, for example, adhesive tape, transparent polyurethane film tape, silicone-based adhesive tape that provides repositionability and gentle removal characteristics, elastic adhesive tape, hydrocolloid adhesive tape, foam tape with pressure-sensitive adhesive backing, nonwoven fabric tape or the like.

In embodiments, the bladder 12 can be positioned within a heel cup 30 or similar anatomical containment structure that exists as part of the cast geometry. The heel cup 30 can be the concave portion of the cast that can conform to and contain the posterior aspect of the patient's heel. When the bladder 12 is inflated to its working pressure before or during the cast setting process, the expanded bladder can form a protective pocket 28 around the patient's heel or other body part being protected as shown in FIG. 5. The bladder 12 can be inflated before the cast sets wherein the bladder forms a pocket 28 around the patient's heel 22 after the cast sets as shown in FIG. 5, allowing for sufficient gap between the skin and the internal casting surface during casting. After the cast is set the bladder 12 can be deflated, wherein the gap can remain without any fluid medium pressure preventing contact between the cast material and the heel. After deflation, the bladder 12 can be removed from the pocket 28 or it can be left the pocket. The pocket 28 can prevent contact between the cast 20 material and the patient's heel or other body part, thereby reducing the risk of pressure sores. In certain embodiments, the bladder 12 can be removed from the pocket 28 wherein the bladder can be removed from a filling port aperture 24.

The pocket 28 can be controlled by adjusting the inflation pressure and volume of the bladder 12 during the casting procedure. The pocket depth measured perpendicular to the skin surface can range from such as, for example, approximately 3 mm to 45 mm depending on the patient's specific anatomy, the severity of pressure sore risk, and the amount of clearance deemed necessary by the healthcare provider. The pocket 28 can extend around the heel in multiple directions, potentially covering the posterior heel surface, the plantar heel surface, and portions of the medial and lateral heel surfaces, depending on the bladder geometry and inflation extent. After the cast 20 material has been applied around the inflated bladder 12 and has achieved sufficient structural rigidity through the curing or hardening process which typically requires 5 to 30 minutes for plaster casts or 3 to 15 minutes for fiberglass casts, depending on the specific product formulation and ambient temperature/humidity conditions, the bladder 12 can be deflated by releasing the inflation medium through the filling port 16 through a valve 23 or the valve can be omitted. This deflation process can be accomplished by such as, for example, cutting the filling port tube, opening a valve, untying a knot, puncturing the bladder, or the like.

In embodiments, after deflation of the bladder 12, the protective gap or pocket 28 that was created by the inflated bladder persists and remains as a permanent air pocket or void space within the cast structure, even in the absence of continued fluid pressure from the deflated bladder. The pocket 28 serves as a protective cushion of air that prevents direct mechanical contact between the cast material 20 and the patient's heel 22 (or other vulnerable tissue), thereby substantially eliminating or dramatically reducing the risk of pressure sore formation throughout the duration of cast immobilization, which can range from several weeks to several months depending on the nature of the underlying orthopedic condition being treated.

After the bladder 12 has been deflated, the collapsed bladder material can be physically removed from the pocket 28 by pulling it out through the filling port aperture 24 in the cast wall. The filling port aperture 24 is a hole or opening in the cast material through which the filling port 16 extends from the interior of the cast to the exterior environment. This aperture 24 can be sized with a diameter to accommodate passage of the filling port tube itself such as, for example, typically 3 mm to 8 mm in diameter, or alternatively can be sized with a larger diameter such as, for example, 10 mm to 30 mm that permits passage of the entire deflated and collapsed bladder 12 through the aperture for complete removal from the cast interior. Removal of the deflated bladder can reduce the total mass and bulk contained within the cast, which can improve patient comfort, reduce cast weight, and enhance mobility.

In other embodiments, the deflated bladder 12 can be intentionally left within the pocket 28 in its collapsed state, where it remains trapped between the heel and the cast interior but does not interfere with the protective function of the air pocket. Leaving the bladder in place can simplify the deflation procedure and eliminates any concerns about damaging the cast structure during bladder removal. The collapsed bladder material is thin and flexible enough that it does not create problematic pressure points or discomfort for the patient.

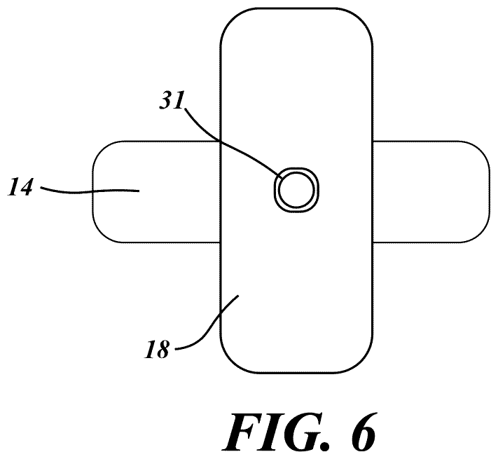

In embodiments, the filling port 16 can be configured to facilitate the controller entry of fluid into the bladder 12. The filling port 16 can be coupled to the bladder 12 and can comprise a valve 23 to prevent backflow ensuring retention of the fluid until the cast 20 sets and the physician is ready to release the fluid. The filling port 16 can extend outwardly from the filling port aperture 24 wherein the filling port aperture 24 can be sized for just the filling port or sized to for the bladder 12 to be removed from inside the pocket 28. The filling port 16 can be coupled to the bladder 12 by such as, for example, thread, adhesive, weld, heat seal, or the like. The filling port 16 can be cut off at the cast, from outside the cast, after the cast is set and the bladder 12 is emptied. In other embodiments, the valve can be omitted from the filling port 16 and the filling port can be sealed by such as, for example, heat seal, crimp, simply knotted like a simple rubber balloon, or the like, at its end and then cut when the fluid needs to be released. In certain embodiments, the filling port 16 can be a rigid structure such as a rigid tube or a rigid ring 31, as shown in FIG. 6, can surround the tube or can be set within the cast forming a hole for the filling port to exit out of allowing for the bladder and filling port to easily pull through the hole and to not stick to the cast after the bladder is emptied or popped from the outside.

In certain embodiments, the cast pressure relieving system 10 can further have a position indicator strip 32 that provides real-time visual feedback regarding the anatomical positioning of the patient's body part within the cast during the critical casting and curing procedures. This indicator strip 32 is typically applied to an appropriate surface of the patient's anatomy before or during the casting process. For heel applications as shown in FIGS. 1-2 , the indicator strip 32 can be adhesively applied to the plantar (bottom) surface of the patient's foot, generally along the midline or slightly to one side. The indicator strip 32 can include graduated visual markings or measurement indicators that are arranged along the length of the strip in a manner similar to a ruler, measuring tape, or other linear measurement device. These graduated markings can be calibrated in various measurement systems including such as, for example, imperial units (inches with fractional or decimal subdivisions), metric units (millimeters, centimeters), or both systems simultaneously on opposite edges of the strip. The markings can be printed, embossed, engraved, or otherwise applied to the strip surface using techniques that ensure the markings remain visible and legible throughout the casting procedure despite exposure to moisture from wet cast materials.

The indicator strip 32 can be dimensioned and positioned such that a portion of the strip extends distally out of the cast margin past the patient's toes (in foot applications) or past other relevant distal anatomical landmarks (in applications to other body parts). The protruding portion of the indicator strip remains visible to the healthcare provider throughout the entire casting procedure and during the cast curing period. The visible graduated markings on the protruding portion provide quantitative feedback about the position of the foot (or other body part) relative to the cast structure. The position indicator strip 32 can be enable the healthcare provider to monitor and precisely adjust the foot's position in real-time as the inflatable bladder 12 is being inflated and as the cast material is being applied and shaped. For example, if the provider observes that the graduated markings have shifted distally (toward the toes), this indicates that the heel has translated proximally (backward) within the cast, suggesting that bladder inflation may need to be adjusted or that the foot position should be manually corrected. Conversely, if the markings shift proximally (away from the toes), this indicates distal heel translation that may also require intervention. This continuous visual monitoring capability helps ensure that the patient's foot maintains its optimal position throughout the cast setting process, which is essential for ensuring uniform pressure distribution, proper immobilization of the fracture or deformity being treated, and effective functioning of the protective pocket 28 created by the bladder system.

The indicator strip 32 can be fabricated from various materials suitable for this application, such as, for example, flexible plastic films, polyethylene, polypropylene, polyester films, silicone sheet materials, or the like. The strip typically has dimensions of such as, for example, approximately 100 mm to 300 mm in length, approximately 5 mm to 25 mm in width, and approximately 0.1 mm to 2 mm in thickness, though these dimensions are exemplary and can be adjusted based on the specific application and patient size. One surface of the indicator strip 32 the surface that contacts the patient's skin can be provided with a medical-grade pressure-sensitive adhesive backing that enables secure attachment to the skin while permitting removal without excessive trauma or pain. The adhesive can be similar to those described previously for the wing attachments and can include a removable release liner that protects the adhesive until the moment of application. The opposite surface of the strip the externally visible surface displays the graduated markings and can be treated with a waterproof or moisture-resistant coating to prevent degradation when exposed to wet plaster or fiberglass casting materials.

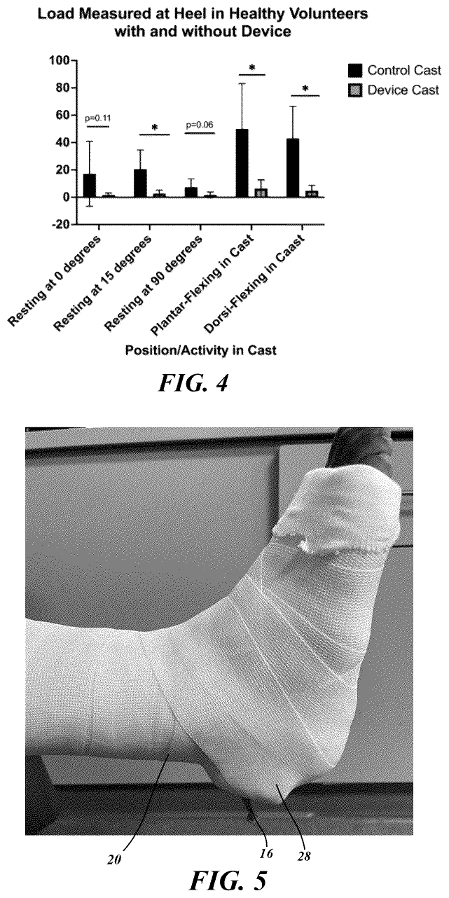

Referring to FIG. 3 which shows a heel load, after the bladder is deflated, on a well-fitted cast with different interventions wherein the cast pressure relieving system is shown as the device wherein the heel load is significantly less than bare leg, no intervention, additional padding, Webril roll felt heel, or the like. FIG. 4 shows a load measured at the heel in healthy volunteer with and without the cast pressure relieving system wherein the loads during position and activity within the cast is shown to be significantly less with the cast pressure relieving system showing the device will help mitigate pressure sores on the patient.

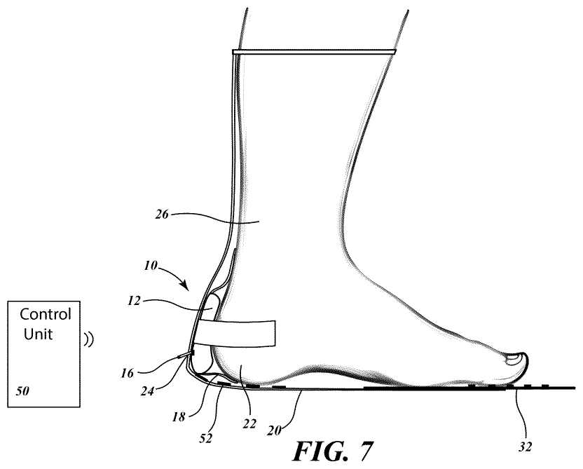

Referring back to FIG. 1-2 and 7, in certain embodiments, the cast pressure relieving system 10 can include a pressure detection system which can include a plurality of sensors 52 positioned in an array around, near or on the patient's high-pressure areas. The pressure detection system 10 can comprise pressure-sensitive materials or sensors embedded within the device or cast. The sensors 52 can be configured to detect high-pressure areas and communicate with a central control unit or mobile device application, which can allow for real-time monitoring of pressure distribution, enabling caregivers to make necessary adjustments to prevent the formation of pressure sores. The sensors 52 can be such as, for example, pressure sensors, capacitive pressure sensor, temperature sensors, optical pressure sensor, flex sensor, humidity sensor, or bacteria or infection sensor, or the like. The pressure detection subsystem can be configured to detect when measured pressure at any sensor 52 location exceeds the programmed threshold value, and to communicate this high-pressure condition to a central control unit or processing device that implements the alert and data management functions. This communication can be accomplished through various wired or wireless communication protocols selected based on range requirements, power consumption constraints, regulatory considerations, and compatibility with existing healthcare information systems.

The central control unit that receives and processes sensor data can be implemented in various hardware configurations. In some embodiments, the control unit can be a dedicated electronic device specifically designed for this cast monitoring application, housed in a small enclosure that can be attached to the exterior of the cast, worn on the patient's clothing, or positioned on a bedside table. The control unit can include a microcontroller or microprocessor for data processing, memory for data logging, a power source such as, for example, replaceable or rechargeable batteries, communication interface components, and user interface elements (such as LED indicators, an LCD display, or buttons. In other embodiments, the control unit functionality can be implemented within a mobile computing device such as a smartphone or tablet computer, with a dedicated software application executing on the device's operating system to receive sensor data, analyze pressure patterns, generate alerts, and provide user interface functions for caregivers.

In other embodiments, the cast pressure relieving system can have cast tube that can be cast around forming a cast hole, such that the tube can connect the inner cavity of the cast where the foot is, to the outside. The cast tube can provide access for a piercing pin to be inserted to the cast hole which can pop the bladder inside the cast after casting, thus creating the cavity behind the heal separating the heal from the inside wall of the casting. In yet another embodiment, the bladder inflation port and narrow neck can themselves be passed from inside the cast to the outside, or the cast is cast around the neck of the balder, leaving its end or port outside the cast. Once the cast cures and is solidified, the balder port neck is simply cut from outside the cast thus allowing total deflation of the bladder inside the cast, which can create the necessary cavity separating the heal skin from the insider of the cast and reducing the risk of pressure sores developing.

The method for mitigating pressure sores during cast immobilization involves positioning the inflatable bladder adjacent to the patient's area of high pressure such as the heel and secured to the patient with at least one wing. The bladder can then be inflated, before placement, or after placement to a custom size correlating to the patient's anatomy. The cast or splint material can be applied around the inflated bladder leaving a aperture for a filling port. The physician can fine tune the bladder and can see these adjustments relative to the cast by indicator strip. This strip provides a visual guide for caregivers, ensuring the foot is correctly positioned within the cast, further reducing the risk of pressure sores. Once the cast is set, the bladder can then deflate, creating an air pocket around the high-pressure area and elevating the high-pressure area to prevent contact with the cast.

The system can also include a control unit 50 in communication with the pressure detection materials. The control unit 50 can be configured to alert caregivers upon detection of excessive pressure, ensuring immediate action can be taken to prevent the formation of pressure sores. The control unit can communicate with a mobile device application for remote monitoring, providing flexibility and convenience for caregivers. The inflation volume and pressure can be adjusted to achieve the desired protective pocket dimensions while maintaining patient comfort. Typical inflation pressures can range from such as, for example, approximately 5 kPa to 100 kPa depending on bladder size, material properties, and the amount of clearance needed between skin and cast. The provider may palpate the inflated bladder to assess firmness and proper positioning. If a valve is present in the filling port, it automatically retains the inflation medium after the inflation device is disconnected. If no valve is present, the port is sealed through heat sealing, crimping, or knotting as described previously.

Concurrently with or following bladder positioning and inflation, the healthcare provider can optionally apply the adhesive indicator strip 32 to the sole of the patient's foot or other appropriate anatomical surface. The strip is positioned such that its graduated markings extend distally beyond the anticipated cast margin past the toes in foot applications, ensuring that a portion of the marked strip will remain visible outside the cast throughout the procedure. The strip adhesive is pressed firmly against the skin to achieve secure bonding. The visible markings establish a reference baseline for monitoring subsequent positional changes.

The method continues with application of cast or splint material around the inflated bladder according to standard orthopedic casting techniques. The cast material which can be plaster of Paris, fiberglass casting tape, or other rigid orthopedic casting materials can be wrapped around the limb in overlapping layers, carefully molding the material to conform to the anatomical contours while accommodating the expanded volume of the inflated bladder. The provider ensures that the cast material does not directly compress or collapse the inflated bladder, which would defeat its protective purpose. The filling port neck is routed through the cast layers such that the port or its terminal end extends outside the cast boundary through the filling port aperture 24, maintaining access for subsequent deflation. If a rigid ring or cast tube is used, it is positioned and embedded appropriately during this casting step.

Fine-tuning of the bladder inflation state can be performed if the pressure sensing system (if present) indicates suboptimal pressure distribution, or if clinical assessment suggests adjustment is needed. Fine-tuning can involve either increasing inflation by pumping additional air or fluid into the bladder through the filling port 16 to increase protective clearance, or decreasing inflation by releasing some fluid from the bladder through the filling port to reduce excessive clearance that might compromise immobilization stability. These adjustments can be made at any point before the cast achieves full rigidity, with the indicator strip providing feedback about the effects of adjustments on limb position.

After the cast material has been fully applied and the healthcare provider is satisfied with the cast configuration, the cast is allowed to cure undisturbed until it achieves complete structural rigidity and can safely bear the mechanical loads associated with patient activity. For plaster casts, full structural cure typically requires 24-72 hours although functional rigidity sufficient for protected weight-bearing is often achieved within 1-2 hours. For fiberglass casts, functional rigidity is achieved more rapidly, typically within 20-30 minutes, although full strength development may take several hours. The provider can assess cure status through palpation, observing surface temperature (curing plaster generates heat), or using clinical judgment based on experience with the specific casting materials employed.

Once the healthcare provider has confirmed that the cast has achieved adequate structural rigidity to maintain its shape without external support, the method proceeds to the critical deflation step. The bladder 12 can be deflated through the filling port 16 by one of several means depending on the specific embodiment being used such as, for example, cutting the filling port tube with scissors or a blade, allowing rapid escape of inflation medium through the severed opening, untying a knot that was used to seal the port after inflation, opening or activating a valve mechanism to permit controlled fluid egress, puncturing the bladder through a cast tube channel using a piercing implement, or the like.

The deflation process can be rapid or more gradual which can take several minutes for liquid-filled bladders with small drainage apertures. If a colored fluid medium was used for inflation, the healthcare provider can observe the fluid draining from the port to confirm complete deflation. For air-filled bladders, deflation can be confirmed by palpating the cast exterior over the bladder location to verify absence of firmness or pressure. The deflation step creates a persistent air pocket or void space around the high-pressure anatomical area, such as the heel, which effectively elevates and protects the vulnerable tissue from contact with the hard internal surface of the cast material. This protective air pocket remains stable throughout the entire cast wearing period, which can extend from weeks to months depending on the underlying condition without requiring any continued inflation pressure or maintenance. The collapsed bladder material may remain within the pocket or may be removed through the filling port aperture, depending on the embodiment and provider preference.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

We claim:

1. A heel pressure relieving system for a patient receiving a rigid cast formed of casting materials in a casting procedure, the system comprising:
a bladder having an extended filling port, an interior cavity and an exterior surface;
at least one wing coupled to the exterior surface of the inflatable bladder wherein the at least one wing is configured to removably affix the inflatable bladder to a skin surface of a body region of the patient;
wherein the extended filling port allows the bladder to receive a fluid to selectively inflate the inflatable bladder from a collapsed configuration to an expanded configuration and allows a user to release the fluid through the filling port after the cast material has cured to form the rigid cast.

2. The device of claim 1, wherein the inflatable bladder is made from material selected from the group consisting of rubber, latex, thermoplastic polyurethane, neoprene rubber, butyl rubber, and nitril.

3. The device of claim 1, wherein the bladder is an elongated shape configured to conform to a body region of the patient.

4. The device of claim 1, wherein the at least one wing member comprises at least one vertical wing and at least one horizontal wing, each wing configured to removably couple to the patient's skin through an adhesive backing.

5. The device of claim 3, wherein the body region comprises a location selected from the group consisting of a heel, an ankle, an elbow, a knee, a head of a fibula, a forearm, and a sacrum.

6. The device of claim 1, wherein the fluid medium comprises at least one material selected from the group consisting of air, water, alcohol, gel, nitrogen, and carbon dioxide.

7. The device of claim 1, further comprising an indicator strip configured to be applied to a surface of the patient's body, the indicator strip having graduated visual markings that provide a visual indication of the body region's position relative to the rigid cast.

8. The device of claim 1, wherein the filling port comprises a valve configured to retain the fluid medium within the interior cavity during the casting procedure and to permit controlled release of the fluid medium after the cast material has cured.

9. The device of claim 1, wherein the extended filling port comprises a tubular neck section extending from the inflatable bladder, the tubular neck section configured to extend through an aperture in the rigid cast to provide external access for inflation and deflation.

10. The device of claim 9, further comprising a rigid ring structure about the tubular neck section configured to be embedded in the cast material during curing, the rigid ring structure forming a defined aperture through which the inflatable bladder can be removed after deflation.

11. The device of claim 1, further comprising a protective cover encapsulating at least a portion of the exterior surface of the inflatable bladder, the protective cover comprising a material selected from the group consisting of nylon fabric, polyurethane-coated fabric, vinyl coating, polyester fabric, mesh material, spandex, and combinations thereof.

12. The device of claim 1, wherein the fluid is colored to provide a visual indicator of the configuration of the bladder.

13. A system for mitigating pressure sores in a patient subjected to cast immobilization, the system comprising:
a bladder configured to form a protective air pocket around a body region of the patient within a cast structure;
a plurality of pressure sensors embedded within or adjacent to the inflatable bladder, the plurality of pressure sensors configured to detect pressure levels between the patient's skin and the cast structure; and
a control unit in communication with the plurality of pressure sensors, the control unit configured to receive pressure data from the plurality of pressure sensors, compare the pressure data to a predetermined pressure threshold and generate an alert when the pressure data exceeds the predetermined pressure threshold.

14. The system of claim 13, wherein the control unit communicates with a mobile device application through a wireless communication protocol selected from the group consisting of Bluetooth, Wi-Fi, cellular communication, and proprietary radio frequency protocols.

15. The system of claim 14, wherein the plurality of pressure sensors comprises sensors selected from the group consisting of capacitive pressure sensors, resistive pressure sensors, piezoelectric sensors, piezoresistive sensors, optical pressure sensors, and combinations thereof.

16. A method for mitigating pressure sores during cast immobilization of a patient, comprising the acts of:
securing an inflatable bladder to a skin surface of a body region of the patient using at least one adhesive wing;
inflating the inflatable bladder with a fluid medium to create an expanded configuration during a casting procedure;
applying cast material that cures to achieve structural rigidity around the inflatable bladder in the expanded configuration;
allowing the cast material to cure and achieve structural rigidity; and
deflating the inflatable bladder after the cast material has cured, thereby creating a persistent air pocket between a rigid cast structure and the skin surface.

17. The method of claim 16, wherein deflating the inflatable bladder comprises an action selected from the group consisting of cutting a filling port of the inflatable bladder, opening a valve mechanism, puncturing the inflatable bladder through a cast tube, and untying a knot sealing the filling port.

18. The method of claim 16, further comprising applying an adhesive indicator strip to the skin surface of the patient before applying the cast material, the indicator strip having graduated markings to provide visual monitoring of a position of the body region during the casting procedure.

19. The method of claim 16, further comprising adjusting an inflation state of the inflatable bladder after cast material has been applied but before the cast material achieves structural rigidity by adding additional fluid medium to increase inflation and/or releasing fluid medium to decrease inflation.

20. The method of claim 16, further comprising removing the inflatable bladder through an aperture in the rigid cast structure after deflation.
